(19) United States
(12) Patent Application Publication
(10) Pub. No.: US 2007/0173460 A1
(43) Pub. Date: Jul. 26, 2007

(54) Compositions comprising lignin and methods of making and using the same

(75) Inventor: Hojabr Alimi, Santa Rosa, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Oculus Innovative Sciences, Inc., Petaluma, CA (US)

(21) Appl. No.: 11/725,279

(22) Filed: Mar. 19, 2007

Related U.S. Application Data
(62) Division of application No. 10/380,902, filed on Mar. 20, 2003, now abandoned.

(51) Int. Cl. A61K 31/70

(52) U.S. Cl. 514/22

(57) ABSTRACT

Lignins have a number of bioactivities including the inhibition of cellular proliferation and inhibition of thrombus formation which are applicable for their use in coating for medical devices and pharmaceuticals. As such, a composition comprising lignins and methods for making and using the same are provided herein.

### CROSS-REFERENCE TO RELATED APPLICATIONS

This patent application is a divisional of co-pending U.S. patent application Ser. No. 10/380,902, filed Mar. 20, 2003, which is a national phase of PCT/US01/29661, filed on Sep. 20, 2001. This patent application further claims the benefit of U.S. Provisional Patent Application No. 60/327,558, filed on Sep. 20, 2000. PCT/US01/29661 and U.S. Patent Application Nos. 60/327,558 and 10/380,902 are hereby incorporated by reference in their entireties.

### TECHNICAL FIELD

This invention is related to compositions comprising lignins or lignin derivatives, and more particularly, to coatings for medical devices and pharmaceuticals comprising lignins such as lignosulfonate having bioactive activity such as the inhibition of cellular proliferation activity and, more particularly, the inhibition of cancer cells in vitro and in vivo.

### BACKGROUND

Lignins are derived from an abundant and renewable resource: trees, plants, and agricultural crops. Commercial lignin is currently produced as a co-product of the paper industry, separated from trees by a chemical pulping process.

Industry first began to use lignins in the 1880 s when lignosulfonates were used in leather tanning and dye baths. Since then, lignins have found applications in food products, serving as emulsifiers in animal feed and as raw material in the production of vanillin. Lignin uses have expanded and it is currently generally used as a binder, dispersant, emulsifier, or sequestrant (for complexing).

While lignins are known in the art they have not been widely used in the biomedical arena. Provided herein for the first time are bioactive activities of lignins and methods of making and using compositions comprising lignins having bioactive activities, particularly, for example, for use in the inhibition of cellular proliferation.

### DISCLOSURE OF THE INVENTION

Compositions comprising a lignin or a lignin derivative and methods of making and using the same are provided herein. The compositions and methods have a number of applications including coatings on medical devices and pharmaceuticals for use in treating cancer, restenosis and thrombosis and in preventing implant rejections and secondary infections.

In one embodiment, a coated medical device having a coating consisting of lignin or a functional derivative thereof is provided. In a preferred embodiment, the lignin is lignosulfonate. The device can be used in vivo such as, but not limited to, a device selected from the group consisting of sutures, bone screws, nails, plates, tubes, sheets, films, stents, artificial valves and vessels, infra-aortic balloons, and prosthetics.

Also provided herein is a method of forming a medical device, said method comprising the step of coating a medical device with a coating comprising lignin or a functional derivative thereof. Preferably, the lignin is lignosulfonate. The coating can be applied in any of a number of suitable ways, for example, but not limited to, dipping, spraying or painting.

In another aspect of the invention, a method of inhibiting restenosis is provided. In one aspect the restenosis is of the type usually caused by tissue response to an inserted and/or implanted medical device. The method comprises the step of coating a medical device with a coating comprising lignin or a functional derivative thereof in an amount effective to inhibit cellular proliferation in vivo, and using said medical device having said coating in vivo. In another embodiment, a composition comprising a lignin or lignin derivative and a pharmaceutically acceptable carrier is administered to a cell, individual or a site in need of inhibition of restenosis in an amount effective for said inhibition. Preferably, the lignin is lignosulfonate.

In yet another aspect of the invention, a coating for a medical device is provided, the coating selected from the group consisting of lignin and functional derivatives thereof. Preferably, the lignin is lignosulfonate.

Also provided herein is a composition comprising lignin or a functional derivative thereof and a pharmaceutically acceptable carrier, wherein the lignin has anti-cellular proliferation activity. Preferably, the lignin is lignosulfonate.

In a further aspect of the invention, a method for inhibiting cellular proliferation is provided which comprises the step of administering to a cell a lignin or a functional derivative thereof. Preferably, the lignin is lignosulfonate. The cell may be in vivo or in vitro. The cell may have a proliferation disorder, including, but not limited to cancer. Preferably, the lignin or a derivative thereof is administered with a pharmaceutical acceptable carrier. Additionally, the pharmaceutical acceptable carrier may further comprise a ligand which targets diseased cells such as cancer cells or virally infected cells such as HIV infected cells.

Other embodiments will be apparent by the detailed description which follows.

### BRIEF DESCRIPTION OF THE DRAWINGS

FIG. 1 shows human smooth muscle cells, more specifically normal human smooth muscle cells as a control;

FIG. 2 shows normal human smooth muscle cells with 2.5% lignosulfate wherein no growth is indicated;

FIG. 3 shows human long fibroblast carcinoma cells, more particularly long fibroblast carcinoma cells as a control having 40% monolayer and 60% tumor cells;

FIG. 4 shows long fibroblast carcinoma cells treated with lignosulfate having 20% tumor cells and 40% monolayer;

FIG. 5 shows the results of an ANOVA used to evaluate the statistical significance of raw data collected in an in vivo study of the effects of L3 on tumor cell development;

FIG. 6 is a scatter diagram showing tumor growth measurements made of mice that received injections of a 7.5% L3 solution in the in vivo study of the effects of L3 on tumor cell development; and

FIG. 7 is a scatter diagram showing tumor growth measurements of control mice in the in vivo study referred to in the brief description of FIGS. 5 and 6.

### BEST MODE FOR CARRYING OUT THE INVENTION

As further described below, it has been discovered herein that lignins and derivatives thereof have a number of bioactivities useful in the biomedical arena. Accordingly, provided herein are compositions comprising lignin or a derivative thereof and methods of making and using the same.

The compositions provided include coatings for medical devices, medical devices with coatings as described herein, and pharmaceuticals comprising lignin or a derivative thereof.

Lignins include lignosulfonates, also called lignin sulfonates and sulfite lignins, and Kraft lignins, also called sulfate lignins. Lignosulfonates are products of sulfite pulping and are hydrophilic. Kraft lignins are obtained from the Kraft pulping process and are hydrophobic. Other delignification technologies use an organic solvent or a high pressure steam treatment to remove lignins from plants are used as is known in the art. In one embodiment, lignosulfonates are used. In another embodiment, Kraft lignins are used.

Commercial lignin is currently produced as a co-product of the paper industry, separated from trees by a chemical pulping process. Generally, trees become logs and bark, all of which goes into chip silos and then through pulping, neutralizing, and evaporating processes, and then to a processing lignin plant where lignin products are produced. Lignins are produced by a variety of companies such as Lenox Polymers Ltd., based in Port Huron, Mich., USA, Tembec, Inc. in Teemiscaming, Quebec, Lignotech, and Georgia Pacific.

In one embodiment, lignosulfonate is prepared by a conventional ethanol precipitation method from a commercially concentrated waste liquor (about 50% of solids concentration) such as Copartin™. Briefly, Copartin™ can be diluted with a 5-fold excess of water, for example, and the solution added dropwise into a 10-fold excess of ethanol (for example). The precipitate is collected by centrifugation and then freeze-dried for further use. In one embodiment, the sugars are removed, for example, by hydrolyzing with sulfuric acid. This method is generally used for sugar composition analysis. Generally, 25 mg of lignosulfonate are dissolved in 0.5 ml of 72% (w/w) H₂SO₄ solution and digested at 30° C. for an hour. After addition of 19.5 ml of water to the mixture it can be autoclaved and dialyzed using standard techniques to obtain sugar free lignosulfonate.

In one embodiment, the lignins are obtained from natural resources. The natural lignins may be modified to include or contain metals, halogens, salts, preferably divalent, or ammonium. Lignins containing iron are a preferred embodiment and have increased activity. Halogens include chlorine, bromine, fluorine, etc., though chlorine is preferred. Chlorinated lignins may be used in certain embodiments in which toxicity is not a factor, such as when diseased cells are targeted. In preferred embodiments, the lignins have minimal toxicity.

In another embodiment, the lignins are synthetic. For example, synthetic lignins are dehydrogenation polymers of p-coumaric acid, ferulic acid, and caffeic acid.

Generally, the lignins, natural or synthetic have increased activity when treated with a reducing agent such as NaBH₄. If desired, the activity can be decreased by treatment with an oxidizing agent such as eerie ammonium nitrate. When a lignin, natural or synthetic is manipulated to have constituents such as calcium, iron, etc., which are not part of the lignin as extracted or synthesized, the lignin is said to be a lignin-based agent or a derivative thereof. Lignin-derivatives as used herein are functional derivatives in that they retain lignin bioactivities as described herein, and preferably have an improved or, for some reason, desirable quality over the lignin on which the lignin derivative is based on. For example, a certain R group may be replaced on a lignin to increase or adjust specificity, activity, pH optimum, stability, etc.

The lignins or derivatives thereof as used herein are generally 1000-20,000 daltons (D), and preferably are about 8,000 to 15,000, more preferably about 9,000 to 12,000 D, and more preferably about 10,000 D. Generally, higher molecular weight lignins correlate with a purer product, which is preferred.

In preferred embodiments, the lignins or derivatives thereof have one or more bioactivities. In a preferred embodiment, the lignins used herein have anti-cellular proliferation activity. “Anti-cellular proliferation activity.” as used herein is used interchangeably with “inhibition of cellular proliferation activity.” Anti-cellular proliferation activity has a number of applications. For use in conjunction with medical devices used in vivo, anti-cellular proliferation activity inhibits or prevents tissue response reactions to inserted and/or implanted medical devices, which reactions result in tissue build up that can lead to disorders such as restenosis, scarring, rejection of the implant, etc. Moreover, compositions that can be administered as Pharmaceuticals comprising lignin or a derivative thereof can be used to inhibit cellular growth of diseased cells such as cancerous cells, or hyperproliferating cells, as further discussed below. For example, drugs which can be administered for example by injection, are discussed below.

In another embodiment, the lignins and derivatives thereof have “anti-thrombogenic activity,” which, as used herein, is interchangeable with “inhibition of thrombus formation activity.” In yet another embodiment, the lignins and derivatives thereof have “anti-restenosis activity.” In yet a further embodiment, the lignins have anti-viral activity, particularly anti-HIV activity. Generally, lignins can inhibit viral replication as well as infectivity. In yet another embodiment, the lignins have antibacterial activity. In yet another embodiment, the lignins have anti-fungal activity.

The inhibitory or “anti-” activity of lignins as used herein, preferably inhibits the particular biological activity by any detectable amount, and more preferably by at least 30%, more preferably 40%, more preferably 50%, more preferably 70%, more preferably 90%, and most preferably by at least 95%. In one embodiment herein, inhibition is 100%. In one aspect, inhibition is defined as any detectable decrease in a particular biological activity, such as cellular proliferation, restenosis or thrombus formation compared to a control not comprising the lignin-containing composition.

In one aspect of the invention, a coating is provided, wherein the coating comprises lignin or a functional derivative thereof. In a preferred embodiment, a medical device is provided wherein the medical device has a coating thereon comprising lignin or a functional derivative thereof. The medical devices provided are preferably for use in vivo. The medical devices can be for limited use in vivo, such as operating or medical device tools such as catheters, laparoscopes, etc., or for temporary or permanent implant devices. In preferred embodiments, the device is selected from the group consisting of sutures, bone screws, nails, plates, tubes, sheets, films, stents, artificial valves and vessels, intra-aortic balloons, and prosthetics. In another embodiment, a medical device is defined as any material which may have tissue contact. In another embodiment, the medical device may be used for cell screening prior to injection. Moreover, the device may be coated, contain, be composed of, or manufactured from lignin or a derivative thereof.

The coating can be applied to the medical device by covalent or ionic binding to the surface of the medical device. Preferably, the coating is covalently bound to part or all of the surface of the medical device. The lignin can be applied as the coating directly or indirectly. Preferably, the lignin is applied indirectly by first mixing said lignin with a secondary coating, and then applying the secondary coating comprising lignin onto said medical device. The secondary coating preferably allows slow release of the lignins and can be any standard secondary coating such as those used to apply heparin to medical devices, generally comprising coated marbles. Usually, the percentage in a lignin coating is about 10% to 65%, more preferably about 20% to 60%, more preferably about 30% to 50%, and more preferably, the coating is about 30% lignin.

The coatings preferably contain lignin as the active agent and are exclusive of other active agents. In another embodiment, the coatings may contain one or more active agents in addition to lignin. For example, other anti-thrombotic agents may be added such as heparin. The coating can be applied by dipping, spraying, painting, electrostatic spraying, or vapor deposition, and is preferably dried at room temperature at 40 to 60% humidity. The coated device is then generally sterilized. The medical devices with the coatings thereon can then be used for their intended use as well as for the bioactivity of the lignin. Thus, methods of reducing tissue response to medical devices such as implants are also provided. For example, in one embodiment a method of inhibiting restenosis associated with the tissue response usually caused by a medical device is provided by use of a medical device having a coating comprising lignin. In another embodiment, a method of inhibiting thrombosis associated with the tissue response usually caused by a medical device is provided by use of a medical device having a coating comprising lignin. In yet another embodiment, a method of inhibiting secondary infections associated with the tissue response usually caused by a medical device is provided by use of a medical device having a coating comprising lignin.

In yet another embodiment, a method for inhibiting cellular proliferation is provided which comprises administering a lignin to a cell. The cell can be any cell. Preferred cell types are smooth muscle cells, endothelial cells and fibroblasts. In one embodiment, the cell is a diseased cell.

Disease states which can be treated by the methods and compositions provided herein include, but are not limited to hyperproliferative disorders and disorders associated with implants. More particular, the methods can be used to treat, but are not limited to treating cancer, autoimmune disease, arthritis, graft rejection, inflammatory bowel disease, proliferation induced after medical procedures, including, but not limited to, surgery, angioplasty, and the like. In alternative embodiments, restenosis, thrombosis, and viral, such as HTV, bacterial, and fungal infections are treated. Thus, in one embodiment, the invention herein includes application to cells or individuals afflicted or impending affliction with anyone of these disorders. Generally, the medical devices described herein are utilized, or more preferably, pharmaceutical compositions are administered to treat said disorders.

Thus, also provided herein are compositions comprising lignin or a lignin derivative. In one embodiment, said lignin or derivative thereof is administered with a pharmaceutical acceptable carrier. In another embodiment, said lignin or derivative thereof is administered with a pharmaceutical acceptable carrier and a ligand which targets diseased cells.

The individual, or patient, is generally a human subject, although as will be appreciated by those in the art, the patient may be animal as well. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient. In a preferred embodiment, the individual requires inhibition of cell proliferation or inhibition of thrombus formation. More preferably, the individual has cancer, a hyperproliferative cell condition, or a condition which requires control of cell division or tissue response either in vivo or in vitro. Moreover, the compositions can be applied to form animal models or cell lines to be screened for other therapeutics.

The compositions provided herein may be administered in a physiologically or pharmaceutically acceptable carrier to a host. The agents and compositions may be administered in a variety of ways, orally, systemically, topically, parenterally e.g., subcutaneously, intraperitoneally, intravascularly, etc. In one embodiment, the inhibitors are applied to the site of a tumor (or a removed tumor) intra-operatively during surgery. Depending upon the manner of introduction, the compounds may be formulated in a variety of ways. The concentration of therapeutically active compound in the formulation may vary from about 0.1-100 wt. %. Generally, a therapeutic amount for the need is used, for example, to achieve inhibition of cellular proliferation, anti-viral, bacterial or fungal activity or anti-thrombogenic activity, among others.

Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The pharmaceutical compositions can be prepared in various forms, such as granules, aerosols, tablets, pills, suppositories, capsules, suspensions, salves, lotions and the like.

Pharmaceutical grade organic or inorganic carriers and/or diluents suitable for oral and topical use can be used to make up compositions containing the therapeutically-active compounds. Diluents known in the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Under certain circumstances it is desirable to provide the inhibitor with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo 15 internalization in cycling, proteins that target intracellular localization and enhance intracellular half-life. The technique of receptor-mediated endocytosis is described, for example, by Wu et al., J. Rial. Chem. 262:4429-4432 (1987); and Wagner et al., Proc. Natl. Acad. Sci. USA 87:3410-3414 (1990). Cell surface markers for cancer cells and HIV infected cells are known in the art and can be targeted using standard techniques.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” in Toxicokinetics and New Drug Development, Yacobi et al, Eds., Pergamon Press, New York pp. 42-96 (1989).

In a preferred embodiment, the methods comprise identifying the inhibitory effect of the lignin or composition comprising lignin. Such assays are standard and known to those skilled in the art. For example, cells can be screened for cytotoxicity and growth inhibition in target tumor (breast, brain and prostate) and control (non tumorigenic) primary cells or cell lines. Cells are placed in 96-well microtiter plates. Next, the lignin compositions are introduced after one day of culture, and are treated for an additional 96 hours. Assays begin at the beginning of drug treatment, at 48 hours and at 96 hours. Qualitative changes are monitored by comparing the amount of cellular protein present at the beginning of the drug incubation period with the amount of protein present in control and test cultures at day 3 and day 5 of growth. Other time points are added as necessary. Quantitative drug-induced changes in culture growth are evaluated using the doubling time and fractional growth rate. See, Skehan, Assays of Cell Growth and Cytotoxicity, G. Studzinski, ed., 2nd Ed., pp 169-191 (1995); Skehan, et al., 1: Natl. Cancer Inst. 82:1107-12 (1990); Skehan, et al., Cell Biol. Toxicol. 2:357-368 (1986).

Additionally, cell populations can be monitored for growth and or viability, often over time by comparing samples incubated with various concentrations of the bioactive agent or without the bioactive agent. Cell number can be quantified using agents such as 3-(4,5-dimethylthiazol-2-yl)-2,5-diphenylteti-azolim bromide (MTT),3-(4,5-dimethylthiazol-2-yl)-5-(3-carboxymethoxyphenyl)-2-(4-sulfophenyl)-2H(MTS)[U.S. Pat. No. 5,185,450] and Alamar Blue which are converted to colored or fluorescent compounds in the presence of metabolically active cells. Alternatively, dyes that bind to cellular protein such as sulforhodamine B (SRB) or crystal violet can be used to quantify cell number. Alternatively, cells can be directly counted using a particle counter, such as a COULTER COUNTER® manufactured by Beckman Coulter, or counted using a microscope to observe cells on a hemocytometer. Preferably, cells counted using the hemocytometer are observed in a solution of trypan blue to distinguish viable from dead cells. Other methods of quantifying cell number are known to those skilled in the art.

It is understood that the examples described below in no way serve to limit the true scope of this invention, but rather are presented for illustrative purposes. All references cited herein are incorporated by reference in their entirety.

### EXAMPLE 1

Lignins Inhibit Cell Growth In Vitro

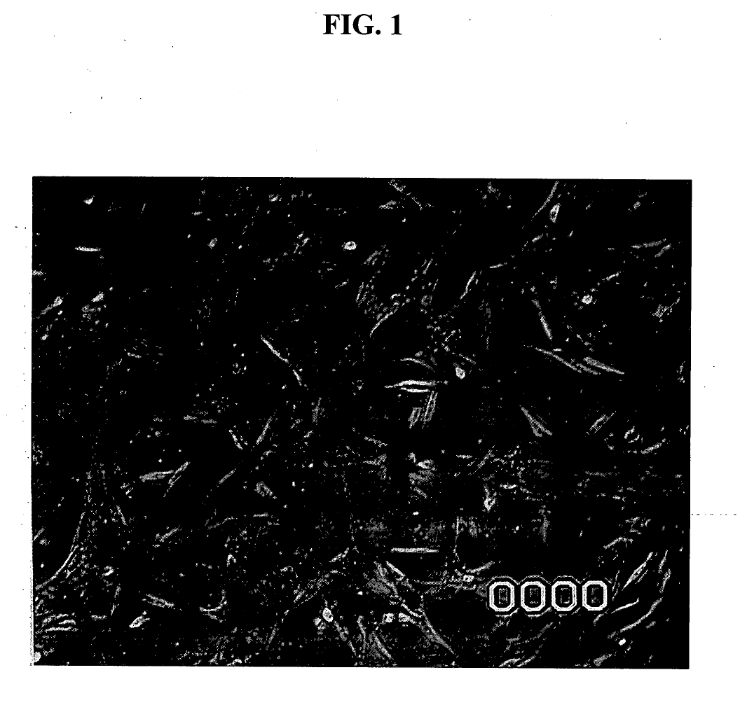

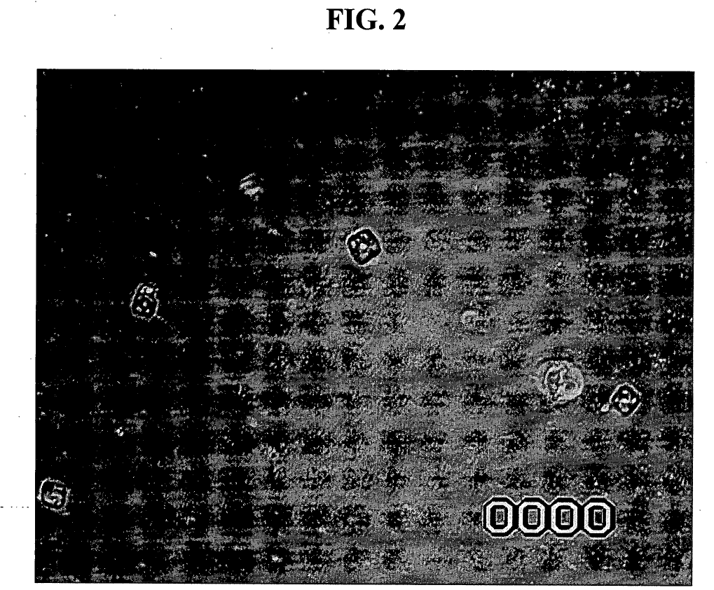

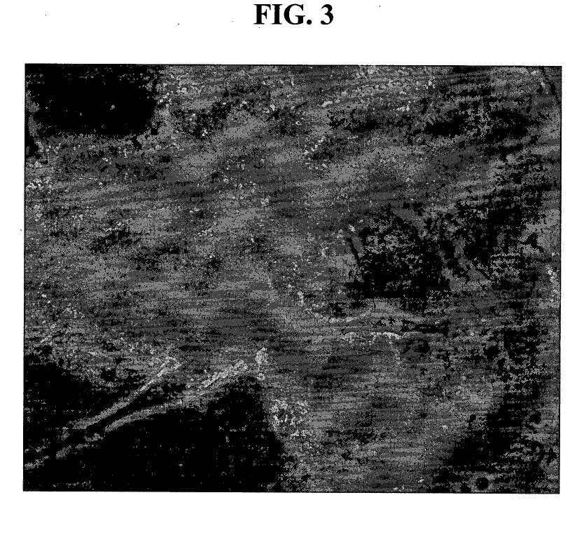

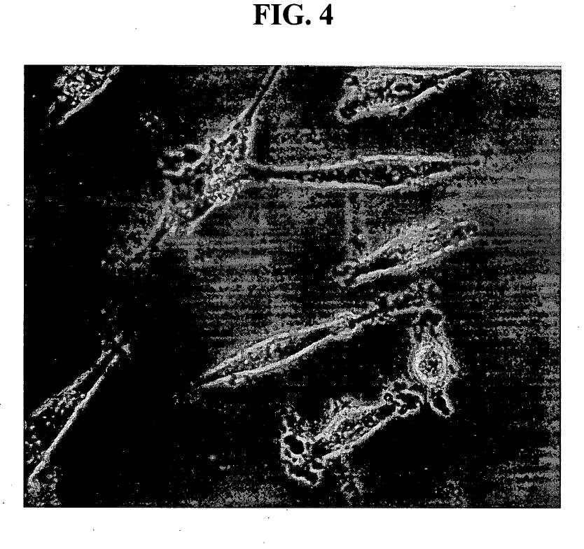

Lignosulfonate was added to human muscle cells in vitro. A 2.5% concentration of lignosulfate was added to the cells which were then incubated for an hour at 37 degrees C. The cells were then harvested and cultured for 20 hours. Cell growth was determined by observing whether cells adhered to the plate and continued to grow and divide. The results indicated that growth in human muscle cells is completely inhibited in vitro when exposed to lignosulfonate. Cancer cells showed similar results. See FIGS. 1-4. Controls were maintained to ensure the integrity of the fat.

As also indicated in FIGS. 1-4, no sign of toxicity was observed. The toxicity was evaluated per ISO 10993 and USP 24. Mouse L929 fibroblast cells were grown in a culture. Once 80%-100% monolayer was achieved, the cells were subjected to 24 and 48 hour exposure to 2.5%, 5% and 10% lignosulfate. The cultures showed no sign of toxicity as stated by USP and ISO. Once again, controls were maintained to ensure the integrity of the fat.

### EXAMPLE 2

Lignins Have Anti-Microbial Activity

Lignosulfonate was added to a variety of microorganisms. The microorganisms were streaked directly onto a media (TSA) containing 2.5% and 5% lignosulfate. Negative control and positive controls were designed to ensure the integrity of the test method. The results indicate that growth was inhibited in Streptomycetes epidermis, Aspergillus niger, and Pemcillium spp. The lignosulfonate did not appeal' to have a noticeable change on Escherichia coir. The inhibition is defined as delay or complete absence of microorganisms to grow on TSA plate containing lignosulfonate.

### EXAMPLE 3

Lignins Inhibit Tumor Cell Development In Vivo

Seventeen mice were received at the age of eight weeks. The mice were maintained and observed for approximately two weeks. Concurrently, melanoma cells specific for C57 mice were maintained and propagated to proper cell cultures. Two 25 cm flasks were used to obtain an appropriate concentration of melanoma cells for injection. A flask containing 24-hour monolayer of cells was removed from the incubator. The monolayer was rinsed with 5 ml of phosphate buffered saline and removed. 3 ml of trypsin was added to the flask and placed in the incubator for 15 minutes±1 minute to allow cells to detach. 5 ml of DMEM was added to the flask and pipetted down the flask wall to remove all cells. Total volume was brought up to 1 Omls with DMEM. The entire volume was transferred to a 50 ml centrifuge tube. The volume of the second flask was transferred to the same tube. The cells were centrifuged for 10 minutes at 125×g. Old supernatant was removed and 10 mls of fresh DMEM was added. A cell count was performed and the cells were spun down again. Supernatant was removed and the cells were suspended in sterile phosphate buffered saline and placed on ice for transport.

Mice were dropped into ajar containing ether soaked cotton balls. Upon sedation (approximately 20 seconds) mice were removed and injected with 0.050 ml of cell suspension. Injections were intradermal on the right flank side. It was noted that the needle pierced through the skin to the outside on two of the injections; accordingly, the needle was withdrawn and the injection site moved to another location. Two mice were not injected and their ears punched with a single hole on the right side. They were maintained as negative controls.

Treatment with L3 began upon confirmation of tumor growth. Once tumors were confirmed, mice were separated into three groups of five. One group of five was maintained as a positive control and had no holes punched in their ears. Another group of five was designated with one hole in the right ear and received treatments with 5% L3. The final group of five mice were designated with a hole in each ear and received treatment with 7.5% L3. Treatment was administered every four days. L3 was prepared and sterilized immediately prior to injections. A new batch was made for every treatment. 0.020 ml of L3 was delivered to the tumor site using a sterile Hamilton syringe. Tumor size was measured using a dissecting ruler. Animals were laid down with their legs outstretched. Tumors were measured along the broadest dimension portion and the diameter of the tumor was recorded in centimeters. Overall health was documented prior to each treatment. Positive control animals were also measured and observed. All observations were documented on technician notes and maintained within the test folder.

Upon completion of treatment, mice were euthanized. One mouse from each test and control group was sampled for histology. Necroscopy was performed on all mice and observations documented.

Results

Mice were confirmed to have a 93.33% uptake of tumors prior to treatment. A single mouse without a tumor was noted and designated null, but this same animal demonstrated a tumor present two days later and was placed in the positive control group.

Positive control mice began dying two days prior to the completion of the study. Tumors were extremely large and systemic infection was noted upon necroscopy. Livers were yellow, spleen was twice the negative size and pancreas were black. Two animals had hair loss on the neck and back and underside of appendages.

Four of the 5% test group died prior to completion of the study. Due to the loss of 5% animals throughout the study, only the 7.5% test group was evaluated statistically. The following results are macroscopic observations obtained throughout the study. Animal #4 died as a result of a relocation. Although there was tumor development, it was small and no infection had set in internally. Animal #4 died before the first treatment. Animal #5 had an open wound at the site of tumor growth as a result of piercing through the skin during delivery. Tumor site had an indentation that had constant bleeding. Treatment fluid leaked out of the wound. The probable cause of death was infection due to a decreased immune response. Animal #2 escaped from its cage and was found dead three days later. The apparent cause of death was dehydration and starvation. There was no discernable enlargement in the tumor and no sign of infection. Animal #1 was choked in its cage lid when another animal was removed for treatment. Its tumor was extremely small (comparable to a grain of sand) and its organs appeared normal. Animal #3 was euthanized at the end of the study. A second tumor was present and organs had a slight sign of infection.

In the 7.5% test group, one animal died prior to completion of the study. Animal #5 had an open wound at the site of cancer cell delivery. The tumor site had an indentation that had constant bleeding. Treatment fluid leaked out the woimd. The probable cause of death was infection due to a decreased immune response. The remaining four animals had tumors that varied in size and various levels in internal infection.

The exposure of treatment schedule for mice included in the study is shown in Table I, below.

TABLE I
Exposure and Treatment Schedule Chronology of In Vivo
Study of the Effects of L3 on Tumor Cell Development

| Date | Action | Comments |
|---|---|---|
| Jun. 27, 2001 | Tumor Cell injection | 5.0 × 10⁵ cells/0.05 ml |
| Jun. 29, 2001 | Relocation | Sutler Medical, |
| | | Sacramento |
| Jul. 9, 2001 | 1st treatment | 14/15 mice had confirmed |
| | | tumors |
| Jul. 11, 2001 | Mouse #4 - 5% died | Stressed from move |
| Jul. 13, 2001 | 2nd treatment | Two mice have open |
| | | wounds |
| Jul. 13, 2001 | Mouse #5 - 5% died | Due to open wound and |
| | | infection |
| Jul. 16, 2001 | Mouse #2 - 5% escaped | N/A |
| Jul. 17, 2001 | 3rd treatment | N/A |
| Jul. 19, 2001 | Mouse #2 - 5% found | Found dead, lack of food |
| | | and water |
| Jul. 19, 2001 | Mouse #5 - 7.5% died | Due to open wound and |
| | | infection |
| Jul. 20, 2001 | 4 m injection | N/A |
| Jul. 20, 2001 | Mouse #1 - 5% died | Choked in cage lid, unable |
| | | to inject |
| Jul. 20, 2001 pm | Positive #3 died | Tumor ruptured, systemic |
| | | infection |
| Jul. 23, 2001 am | Positive #1 died | Tumor ruptured, systemic |
| | | infection |
| Jul. 23, 2001 | Necroscopy | Tumors and organs taken |
| | | for histology |

Analysis

Raw data for the study follows in Table II, below.

TABLE II
Raw Data: Control Group vs. 7.5% Test Group

| Animal | Control | Test | |
|---|---|---|---|
| P-1 | 1.4 | 7.5-2 | 1.2 |
| P-1 | 2.6 | 7.5-2 | 1.4 |
| P-1 | 3 | 7.5-2 | 1.9 |
| P-1 | 3.1 | 7.5-2 | 2 |
| P-2 | 0.6 | 7.5-3 | 0.4 |
| P-2 | 1.9 | 7.5-3 | 0.7 |
| P-2 | 2.6 | 7.5-3 | 1.2 |
| P-2 | 2.8 | 7.5-3 | 1.6 |
| P-3 | 0.8 | | |
| P-3 | 1.8 | | |
| P-3 | 2.7 | | |
| P-3 | 2.4 | | |
| P-4 | 1.1 | | |
| P-4 | 2.4 | | |
| P-4 | 3 | | |
| P-4′ | 3.1 | | |
| P-5 | 0.5 | | |
| P-5 | 1.7 | | |
| P-5 | 2 | | |
| P-5 | 2.1 | | |

Evaluation of the 7.5% test group was conducted. As per the Tumor Growth Table, Table III below, animal numbers 1 and 4 were not analyzed due to the inability to detect variance. Animal #5 was excluded from the analysis because it did not complete the study.

TABLE III
Data and Observations Tumor Growth

Treatment

| | | | |
|---|---|---|---|
| 2nd | 3rd | 4th | Terminate |

| | | |
|---|---|---|
| Animal | Size of tumor in cm | % Increase |

| | | | | | |
|---|---|---|---|---|---|
| P-1 | 1.4 | 2.6 | 3.0 | 3.1 | 121 |
| P-2 | 0.6 | 1.9 | 2.6 | 2.8 | 367 |
| P-3 | 0.8 | 1.8 | 2.7 | 2.4 | 200 |
| P-4 | 1.1 | 2.4 | 3.0 | 3.1 | 181 |
| P-5 | 0.5 | 1.7 | 2.0 | 2.1 | 320 |
| 5-1 | SG | NC | NC | D | 0 |
| 5-2 | 0.7 | D | D | D | 0 |
| 5-3 | 1.2 | 1.4 | 1.9 | 2.1 | 75 |
| 5-4 | D | D | D | D | 0 |
| 5-5 | 0.8 | D | D | D | 0 |
| 7.5-1 | SG | NC | NC | SG | 0 |
| 7.5-2 | 1.2 | 1.4 | 1.9 | 2.0 | 67 |
| 7.5-3 | 0.4 | 0.7 | 1.2 | 1.6 | 300 |
| 7.5-4 | SG | 0.5 | 0.5 | 0.5 | 50 |
| 7.5-5 | 1.1 | 1.2 | D | D | 9 |

Key to Table III: SG—sand grain size, cannot be visualized or measured; D=animal removed from study due to death or escape; NC=no change in tumor size.

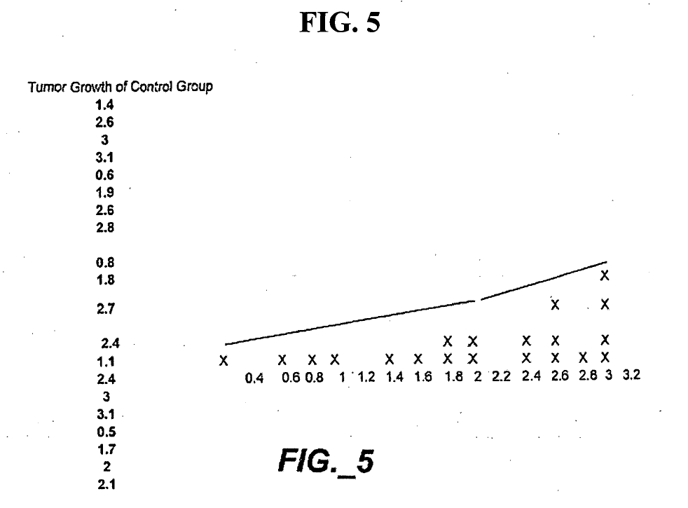

An ANOVA, represented in FIG. 5, was used to evaluate the statistical significance of the data. From the evaluation it may be concluded that L3 inhibits tumor development. This conclusion is based on a p value of 0.023362 and an assumed value of p=0.05.

Animals numbered 1 and 4 within the 7.5% test group showed minimal growth when compared to results from control. The mean increase in tumor size in the control group was 23.8%. This compares to a mean tumor size increase in the 7.5% test group of 104%.

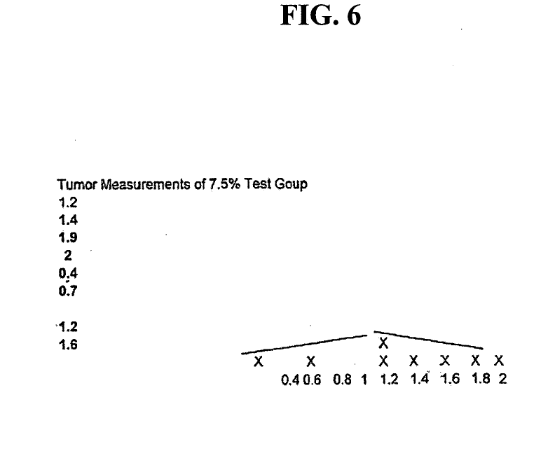

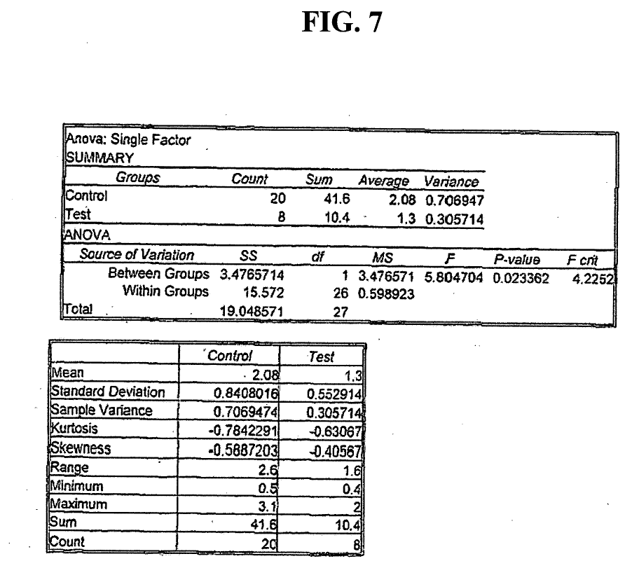

Upon review of scatter diagrams for the raw data, FIGS. 6-7, it is apparent that the tumor growth of the control group continued to increase while the test group demonstrated a limited growth. The control group showed a positive slope and the test group showed a negative slope. From the analysis it can be concluded that the 7.5% concentration of L3 has a reduction in cell proliferation when compared to control animals.

While this invention has been described in connection with preferred embodiments thereof, it is obvious that modifications and changes therein may be made by those skilled in the art to which it pertains without departing from the spirit and scope of the invention. Accordingly, the scope of this invention is to be limited only by the appended claims.

1. A method for inhibiting cellular proliferation comprising the step of administering to a cell a bioactive agent selected from the group consisting of an oxidized lignin and oxidized functional derivatives of lignin.

2. The method of claim 1, wherein said bioactive agent is an oxidized lignosulfonate.

3. The method of claim 2, wherein the lignosulfonate is prepared by ethanol precipitation from concentrated waste liquor.

4. The method of claim 2, wherein the lignosulfonate is obtained from natural resources.

5. The method of claim 2, wherein the lignosulfonate is oxidized with ceric ammonium nitrate.

6. The method of claim 1, wherein the bioactive agent has a molecular weight between 1,000 to 20,000 Daltons.

7. The method of claim 1, wherein the bioactive agent has a molecular weight between 8,000 and 15,000 Daltons.

8. The method of claim 7, wherein the bioactive agent has a molecular weight between 9,000 and 12,000 Daltons.

9. The method of claim 2, wherein the cellular proliferation is of cancerous cells.

10. The method of claim 2, wherein the cellular proliferation is in an individual with cancer.

11. The method of claim 2, wherein the cellular proliferation is of virally infected cells.

12. The method of claim 2, wherein the cellular proliferation is in an individual with a viral infection.
